(19) United States
(12) Patent Application Publication
(10) Pub. No.: US 2025/0241867 A1
(43) Pub. Date: Jul. 31, 2025

(54) HYDROGEL PATCH COMPOSITION USING ARNICA FERMENTED EXTRACT

(71) Applicant: WOOSHIN LABOTTACH CO., LTD., Seoul (KR)

(72) Inventor: Taek Soo NAM, Seoul (KR)

(73) Assignee: WOOSHIN LABOTTACH CO., LTD., Seoul (KR)

(21) Appl. No.: 18/798,837

(22) Filed: Aug. 9, 2024

(30) Foreign Application Priority Data
Jan. 26, 2024 (KR) 10-2024-0012589

(51) Int. Cl. A61K 9/70; A61K 9/00; A61K 36/28; A61K 47/10; A61K 47/32; A61P 29/00

(52) U.S. Cl. CPC A61K 9/7023; A61K 9/0009; A61K 36/28; A61K 47/10; A61K 47/32; A61P 29/00

(57) ABSTRACT

The present invention relates a novel formulation of a hydrogel or cataplasma patch containing an Arnica extract, wherein a composition for a hydrogel or cataplasma patch containing an Arnica extract, includes 30% to 70% by weight of water as a solvent based on the total composition, 20% to 60% by weight of a polyhydric alcohol based on the total composition, and a partially neutralized polyacrylic acid product as a polymer for forming a polyhydric alcohol gel, the composition being effective for suppressing inflammation, alleviating pain or alleviating hot plate pain.

### BACKGROUND OF THE INVENTION

The present invention relates to an anti-inflammatory analgesic composition in the form of a transdermal absorption preparation capable of exhibiting therapeutic efficacy on sprains, bruises, muscle pain, joint pain, fracture pain, back pain, stiff shoulders, neuralgia, rheumatic pain, skin itchiness, insect bites, or chilblains, using an Arnica fermented extract (hereinafter also referred to as an Arnica extract).

### [Formulation Research Trend]

Patches are classified into cataplasma, which is a moisture-containing poultice, and plaster, which is a solid preparation. Due to the nature of the preparation, cataplasmas include a separate adhesive layer with low elasticity in addition to an adhesive layer containing the drug. Plasters, which contain an acrylic resin or the like, are products that do not contain moisture, so they have the disadvantage of being more skin-irritating than cataplasmas.

Hydrogel patches are being studied as a transdermal absorption preparation to overcome the disadvantages of conventional cataplasmas, plasters, or cream-type formulations.

### [Homeopathic Therapy]

The history of homeopathic therapy began with Samuel Hahnemann, an 18th-century German physicist who assumed that “like cures like.” In the 19th century, Hugo Paul Friedrich Schultz postulated that taking a small amount of toxin would have opposite effects compared to taking a large amount of toxin. He demonstrated in 1888 that mild concentrations of yeast toxin increased yeast growth a hundredfold. Thus, these different observations were formulated by Arndt in 1988 as an early pharmacological law of homeopathic effectiveness.

[Arnica montana]

Arnica montana refers to a flower of Arnica montana, a plant of Asteraceae family of Asterales order, and the plant grows in grasslands of highlands and is 20 to 30 cm tall. The leaves emerge from the roots, spread in all directions, and are foliated oppositely each other on the stem. The branches split at the end and have 1 to 3 heads. The flowers bloom in yellow from June to July, are 6 to 8 cm in diameter, and are densely covered with pili and hairs. The flowers and rhizomes have a bitter taste and contain an essential oil and a resin. The flowers and rhizomes were used as an all-purpose folk medicine in Europe since ancient times. In oriental medicine, it is also used for stimulating in angina, dilating blood vessels, relieving vascular spasm, and a hemostatic agent treating bruises and hemorrhoids. Among various organs or parts (e.g. leaves, bark, branches, flowers, roots, fruits, stems, etc.) of the Arnica montana, the whole plants are used in the present invention.

An Arnica extract, which is a purely vegetable homeopathy therapeutic agent containing an Arnica extract, is topically used for pain treatment and not orally administered in generally due to the potential toxicity. Gels and ointments for topical use are available. The active ingredients include sesquiterpene lactones, flavonoids, and volatile oils.

For example, Korean Patent Publication No. 10-1321031 discloses a homeopathic preparation including an Arnica extract, which is a tincture preparation blended with ethanol. There is no mention of a patch. On the other hand, Korean Patent Publication No. 10-2021-0135034 relates to a cosmetic composition for reinforcing the skin barrier, but there is no mention of a patch here either, and a lotion or oil blended with alcohol is disclosed.

### CHARACTERISTICS OF THE PRESENT INVENTION

Among patches, the primary functions of hydrogel patches are mostly the cooling and soothing effects referred to cryotherapy (general use of low temperature in treatment). The primary effect of cold in pain management is to lower the temperature of damaged tissues, which reduces the metabolic rate of the tissues and helps the tissues survive after damage. In addition, the cooling effect may prevent internal bleeding caused by the constriction of peripheral blood vessels.

The Arnica hydrogel cataplasma patch formulation provided by the present invention provides a unique dual action (cool and hot sensations). Just like the heating effect by the stable ether vanillyl butyl ether (VBE), the patch formulation of the present invention directly stimulates the receptors of the nerve endings to create a feeling of heat. There is no actual change in the skin temperature, but instead, the neurotransmitter of the nerve acts to produce a perceived heating effect.

### RELATED ART DOCUMENTS

### Patent Documents

Korean Patent Publication No. 10-1321031
Korean Patent Publication No. 10-2021-0135034

### SUMMARY OF THE INVENTION

The object of the present invention is to provide an anti-inflammatory analgesic composition exhibiting therapeutic efficacy on sprains, bruises, muscle pain, joint pain, fracture pain, back pain, stiff shoulders, neuralgia, rheumatic pain, skin itchiness, insect bites, or chilblains, using an Arnica montana fermented extract.

The present invention develops a hydrogel cataplasma formulation with water as the main component, so that it has excellent adhesiveness, allows a large amount of drug to be penetrated for a long time, and does not affect activities after the patch is attached to the skin.

After gel cream formulations are applied to the skin, clothing or other things are attached to the skin, hindering long-term drug penetration and limiting the patient's activities. Dry patches, the adhesiveness is low because the surface is oily, and especially when the patient's skin pores sweat or water, the adhesiveness completely disappears. This also limits the patient's activities.

However, the present invention achieves the above and other objects by the description below.

In order to achieve the above object, the present invention has the following structural features.
1. A pharmaceutical composition in the form of a hydrogel or cataplasma patch, including: an Arnica extract; a partially neutralized polyacrylic acid product as a hydrogel-forming agent; a combination of an acid and one or more crosslinking agents selected from the group consisting of aluminum salts, magnesium salts, and calcium salts; and 30% to 70% by weight of water (preferably 35% to 60% by weight, more preferably 35% to 50% by weight) based on the total weight of the composition (the Arnica extract may be included in an amount of 0.1% to 5.0% by weight based on the total weight of the composition).
2. The pharmaceutical composition in the form of a hydrogel or cataplasma patch according to Item 1 above, further including at least one selected from the group consisting of ketoprofen or a pharmaceutically acceptable salt thereof, flurbiprofen or a pharmaceutically acceptable salt thereof, lidocaine or a pharmaceutically acceptable salt thereof, and diclofenac or a pharmaceutically acceptable salt thereof.
3. The pharmaceutical composition in the form of a hydrogel or cataplasma patch according to Item 1 or 2 above, wherein the partially neutralized polyacrylic acid product may have a neutralization degree of 25% to 70% by mole, and may be present in an amount of 3% to 8% by weight based on the total weight of the composition.
4. The pharmaceutical composition in the form of a hydrogel or cataplasma patch according to any one of Items 1 to 3 above, wherein the crosslinking agent is at least one selected from the group consisting of a dried aluminum hydroxide gel, dihydroxyaluminum aminoacetate, aluminum hydroxide, aluminum sulfate, aluminum acetate, aluminum silicate, magnesium hydroxide, magnesium sulfate, magnesium acetate, calcium hydroxide, calcium carbonate, calcium chloride, calcium gluconate, calcium lactate, and calcium pantothenate and is present in an amount of 0.1% to 3.0% by weight based on the total weight of the composition.
5. The pharmaceutical composition in the form of a hydrogel or cataplasma patch according to any one of Items 1 to 4 above, further including one or more dispersing agents selected from the group consisting of glycerin, polyethylene glycol, dipropylene glycol, and butylene glycol, wherein the dispersing agent is present in an amount of 20% to 60% by weight based on the total weight of the composition.
6. The pharmaceutical composition in the form of a hydrogel or cataplasma patch according to any one of Items 1 to 5 above, further including one or more additives selected from the group consisting of an acid, a penetration enhancer, a viscosity modifier, a chelating agent, and a fragrance, wherein the acid may be one or more selected from the group consisting of tartaric acid, acetic acid, propionic acid, isobutyric acid, caproic acid, caprylic acid, lactic acid, maleic acid, succinic acid, tartaric acid, pyruvic acid, oxalic acid, phthalic acid, salicylic acid, benzoic acid, acetylsalicylic acid, ethanesulfonic acid, propylsulfonic acid, butanesulfonic acid, and methanesulfonic acid and may be present in an amount of 1% to 10t % by weight based on the total weight of the composition, the penetration enhancer may be one or more selected from the group consisting of isopropyl myristate, lauryl alcohol, propylene glycol monolaurate, oleic acid, polyoxyethylene lauryl ether, diethylene glycol monoethyl ether, and isostearyl glyceryl ether and may be present in an amount of 0.5% to 10t % by weight based on the total weight of the composition, the chelating agent may be edetic acid or a salt thereof and may be present in an amount of 0.2% to 3% by weight based on the total weight of the composition, and the fragrance may be one or more selected from the group consisting of L-menthol, DL-camphor, eucalyptol, and peppermint oil and may be present in an amount of 0.1% to 4% by weight based on the total weight of the composition.

### BRIEF DESCRIPTION OF THE DRAWINGS

The accompanying drawings are intended to explain the contents of the present invention in more details to those skilled in the art, but the technical principle of the present invention is not limited thereto.

FIG. 1 shows a diagram illustrating the inhibitory efficacy of the Arnica gel and patch of the present invention on paw edema in a carrageenan-induced mouse inflammation animal model. (A) of FIG. 1 shows the experimental process. Carrageenan was injected into the right paw of the mouse, and the vehicle was injected into the left paw three times for a total of three days, and the Arnica patch was applied and replaced twice a day. Arnicare Gel® (Boiron Inc., France) was applied three times a day. After carrageenan administration on the third day, the thickness of the paw was measured three times at one-hour intervals to confirm the degree of change in paw edema, and the actual thickness of the paw was photographed at hour 1 and hour 3, and the results are shown in (B) of FIG. 1. These are photographs of edema induction in the vehicle administration group (CRG only), Arnica gel administration group (CRG+Gel), and Arnica patch administration group (CRG+Patch) at hour 1 and hour 3 after the third carrageenan administration on the third day, respectively.

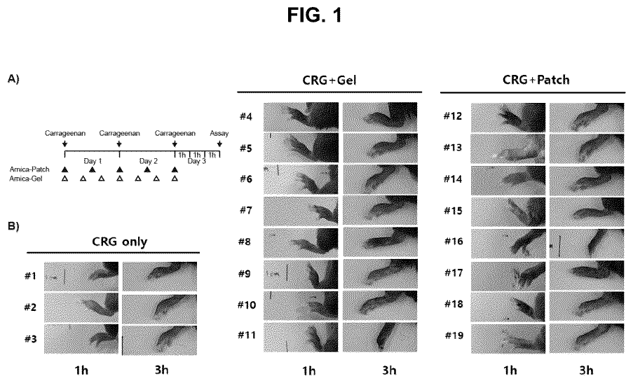

FIG. 2 shows a diagram illustrating the inhibitory efficacy of Arnicare Gel® (Boiron Inc., France) and patches on paw edema formation in a carrageenan-induced inflammation animal model. (A) of FIG. 2) Left, the thickness of the right paw in which edema formation was induced with carrageenan; Right, the ratio of the thickness of the right paw with carrageenan-induced edema to the thickness of the left paw treated with saline (Control). (B) of FIG. 2) Left, the difference in the thickness of the right paw with carrageenan-induced edema to the left paw treated with saline (Control) at different hours; Right, the difference in the thickness of the right paw with carrageenan-induced edema to the left paw treated with saline at hour 3.

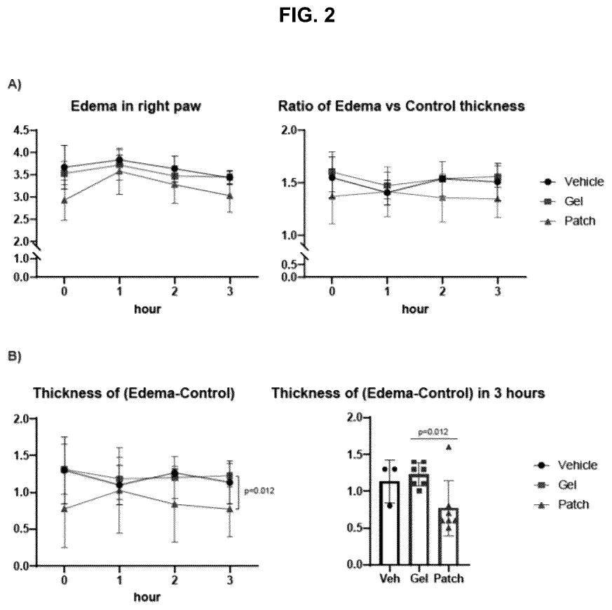

FIG. 3 shows representative photographs of the H&E-stained tissue of the paw edema in the carrageenan-induced inflammation animal model. Compared to the H&E results of the paw that was not administered carrageenan, a wide and weak tissue composition due to the edema was confirmed in the cross-sectional H&E results of the paw in which edema was induced by carrageenan, and it can also be seen that the infiltration of inflammatory cells (neutrophils) was significantly increased in the CRG only group. The inflammatory cell infiltration was clearly suppressed in the Arnicare Gel® (Boiron Inc., France) group. According to the analysis of the tissue photographs of the Arnica patch group, the efficacy was exhibited that the tissue composition became even denser, and the inflammatory cell infiltration was further reduced compared to the other groups.

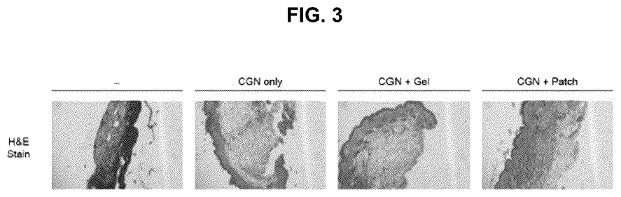

FIG. 4 shows a diagram illustrating the inhibitory effect of the Arnica patch on changes in inflammatory markers in the paw edema in the carrageenan-induced inflammation animal model. The left shows changes in the Tnf nRNA expression level in the tissue of the right paw in which edema formation was induced with carrageenan, the middle shows changes in the II1b mRNA expression level in the tissue of the right paw in which edema formation was induced with carrageenan, and the right shows changes in 116 mRNA expression level in the tissue of the right paw in which edema formation was induced with carrageenan. Representative inflammatory markers, Tnf, II1b, and 116 mRNA, were compared by group.

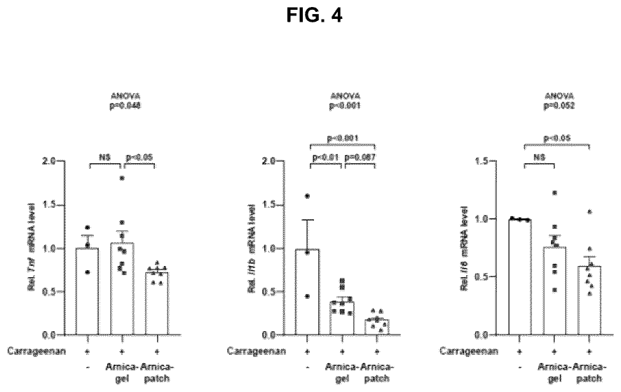

FIG. 5 shows photographs of a mouse animal experiment to confirm sensitivity to pain. The left shows a photograph of a rat's left paw treated with the drug, and the right shows a photograph of a mouse whose movement is restricted by a transparent acrylic cylinder.

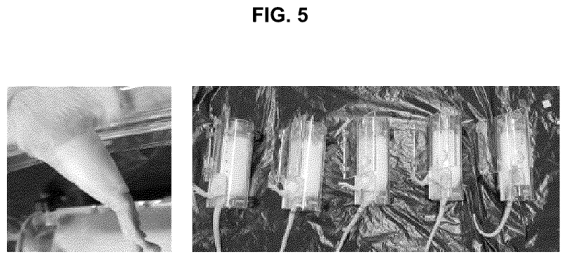

FIG. 6 shows the results of a mouse animal experiment on pain sensitivity. The results are pain scores measured at hour 1 and hour 2 after the control group, hydrogel patch, and gel formulation were treated in a hot plate experiment. Data are expressed as the mean±standard error of the mean (SEM). **p<0.01 vs. Control. (Grouped analysis, Two-way ANOVA).

### DETAILED DESCRIPTION OF THE EMBODIMENTS

### Definition

The term “Arnica extract” herein does not have a particular specifications, and includes, for example, Arnica montana extracts that are commercially available for ISO-certified supplements, foods, beverages, cosmetics, or pharmaceuticals.

The present invention relates to a hydrogel or cataplasma patch formulation. However, it is to be noted that in the following, the hydrogel or cataplasma patch formulation is also sometimes expressed as a gel, hydrogel, hydrogel patch, or patch. For reference, a hydrogel refers to a gel state, and a cataplasma patch refers to a patch in which a hydrogel is applied to a nonwoven fabric, or the like.

The total amount of the base herein refers to the total weight of the composition.

### [Composition]

The present invention provides a pharmaceutical composition in the form of a hydrogel or cataplasma patch including: a pharmaceutically active ingredient; a partially neutralized polyacrylic acid product as a gel-forming agent; a combination of one or more crosslinking agents selected from the group consisting of aluminum salts, magnesium salts, and calcium salts and an acid; and 30% to 70% by weight of water.

### a) Active Ingredient

In the pharmaceutical composition of the present invention, the active pharmaceutical ingredient (API) includes an Arnica extract, and other ingredients that may be combined are not particularly limited. Preferred APIs include anti-inflammatory analgesics, and for example, loxoprofen, flurbiprofen, ketoprofen, lidocaine, diclofenac, or pharmaceutically acceptable salts thereof may be combined with the Arnica extract.

The APIs may be used in their therapeutically effective amounts, including the Arnica extract, and are not particularly limited. For example, the Arnica extract may be included in an amount of 0.1% to 5.0% by weight based on the total weight of the composition.

b) Water-Soluble Polyacrylic Acid and/or its Salt

The pharmaceutical composition of the present invention includes a partially neutralized polyacrylic acid product as a gel-forming agent. When a partially neutralized polyacrylic acid product is used as a gel-forming agent, the API is not precipitated from the hydrogel and may provide excellent stability even when stored for a long period of time. Examples of the partially neutralized polyacrylic acid product may include a commercially available powdered partially neutralized polyacrylic acid product manufactured by Nippon Pure Chemical Industries, Ltd. (trade name “ARONVIS AH-105”), a powdered sodium polyacrylate manufactured by Nippon Pure Chemical Industries, Ltd. (trade name “ARONVIS S”), a polyacrylic acid aqueous solution manufactured by Nippon Pure Chemical Industries, Ltd. (concentration 20% by weight, trade name “JURYMER AC-10H”), and a powdered cross-linked polyacrylic acid manufactured by Nippon Pure Chemical Industries, Ltd. (trade name “JUNLON PW-110”). These may be used alone or in combination of two or more. The amount to be used is preferably 1% to 30% by weight based on the total amount of the base (total composition), and more preferably 3% to 15% by weight.

When the use amount of water-soluble polyacrylic acid and/or a salt thereof is less than 1% by weight, it rarely contributes to improving the skin adhesiveness of the obtained base or the gel strength of the solid. In addition, when it exceeds 30% by weight, there are cases where the hardness and adhesiveness of the obtained base are unbalanced.

### c) Polyhydric Alcohol

The base for the patch of the present invention contains a polyhydric alcohol as an essential component as a dispersing agent, and the polyhydric alcohol also serves as a moisturizing agent or a water-retaining agent. Specific examples of polyhydric alcohols that are suitably used include, for example, glycerin, sorbitol, mannitol, xylitol, ethylene glycol, diethylene glycol, 1,3-propanediol, 1,4-butanediol, polyethylene glycol, dipropylene glycol, propylene glycol, butylene glycol, polypropylene glycol, hexylene glycol, and 1,2-hexanediol. The use amount thereof is usually 1% to 60% by weight, preferably 5% to 60% by weight, more preferably 20% to 60% by weight, and even more preferably 20% to 40% by weight, based on the total amount of the base. When the use amount is less than 1% by weight, the moisture evaporates from the obtained base so that the base is dried, and the drug may precipitate into the base. In addition, when it exceeds 60% by weight, there are cases where the hardness and adhesiveness of the obtained base are unbalanced.

### d) Organic Acid Having a Hydroxyl Group or a Salt Thereof

In the pharmaceutical composition of the present invention, an organic acid having a hydroxyl group or a salt thereof may be used in combination according to the required properties. Examples of the organic acid having a hydroxyl group or a salt thereof, from the viewpoint of skin irritation or safety, may include tartaric acid, acetic acid, propionic acid, isobutyric acid, caproic acid, caprylic acid, lactic acid, maleic acid, succinic acid, tartaric acid, methanesulfonic acid, citric acid, glycolic acid, gluconic acid, pyruvic acid, oxalic acid, phthalic acid, salicylic acid, benzoic acid, acetylsalicylic acid, ethanesulfonic acid, propylsulfonic acid, butanesulfonic acid, and alkali metal salts thereof. Particularly preferred are tartaric acid, citric acid, malic acid, and alkali metal salts thereof. For example, tartaric acid is preferred also in the sense the gel-hardening speed is accelerated by combined use with aluminum hydroxide or aluminum glycinate.

### e) Water

The base for patch of the present invention is characterized in that it is used in an aqueous system. As water, water such as purified water is usually used. The specific amount of water used is determined according to the characteristics required for the patch base, and is usually 10% to 85% by weight. According to an example, water may be included in an amount of 30% to 70% by weight based on the total amount of the composition, preferably 35% to 60t % by weight, and more preferably 35% to 50% by weight.

In particular, when water and a polyhydric alcohol are mixed at a certain content ratio, patch formation is excellent, and an example of this may be a combination of 30% to 70% by weight of water and 20% to 60% by weight of a polyhydric alcohol.

f) Long-Chain Fatty Acid and/or an Ester Thereof

The base for patch of the present invention may further be mixed with a long-chain fatty acid and/or an ester thereof depending on the purpose. This is used as a hydrophobic additive mixed in the base, as a dissolution aid for drugs, or to improve the percutaneous absorbability of an API. As the long-chain fatty acid and/or ester thereof, oleic acid, linoleic acid, linolenic acid, myristic acid, and esters thereof are preferably used in terms of safety and effectiveness.

The use amount thereof is preferably 0.1% to 20% by weight, more preferably 1% to 10% by weight based on the total amount of the base. When the use amount is less than 0.1% by weight, it rarely contributes to improving the solubility of the hydrophobic additive or drug, and in some cases, the additive or drug may be precipitated into the base, or the percutaneous absorbability of the drug may decrease.

### g) Chelating Agent

The chelating agent may be edetic acid or a salt thereof, and may be present in an amount of 0.2% to 3% by weight based on the total weight of the composition.

### h) Other Ingredients

In addition to the above ingredients, the base for patch of the present invention may be mixed with one or more types of additives commonly used in patches, i.e., other ingredients such as a fragrance, an antioxidant, an antiseptic, an emulsifier, an inorganic powder, a penetration enhancer and the like, in order to improve the quality at the time of use.

As these other ingredients, one or more may be selected from the group consisting of fragrances such as of L-menthol, DL-camphor, eucalyptol, peppermint oil, thymol, peppermint oil, castor oil, eucalyptus oil, and lavender oil; warming sensation agents such as a red pepper extract and a prickly ash extract; antioxidants such as butylated hydroxytoluene and butylated hydroxyanisole; preservatives such as methyl parahydroxybenzoate and propyl parahydroxybenzoate; emulsifiers; and inorganic powders such as kaolin, titanium oxide, and anhydrous silicic acid. As the penetration enhancer, one or more may be selected from the group consisting of isopropyl myristate, lauryl alcohol, propylene glycol monolaurate, oleic acid, polyoxyethylene lauryl ether, diethylene glycol monoethyl ether, and isostearyl glyceryl ether.

Specific examples of the mixing ratio of these fragrances to inorganic additives, and the like, are typically 1% to 20% by weight each based on the total amount of the base for patch.

### [Method for Preparing Hydrogel Patch]

The present invention is characterized by the composition, and the method of preparing the base for patch of the present invention is not particularly limited.

Generally, the active ingredient Arnica extract and water are weighed in percentage by weight of the example and stirred (solution A). After dissolving water-soluble polyacrylic acid in water (solution B), sodium edetate and an acid are added to solution B, and the resulting mixture is stirred (solution C) Solution A is added to solution C and mixed (solution D). A separate mixture prepared by dissolving glycerin, a dissolution aid, a penetration enhancer, a colorant, and a viscosity modifier (solution E) is added to solution D, and then dispersed for about 30 minutes. The obtained mixture is deposited between a polyethylene terephthalate film and a nonwoven fabric by adjusting the thickness to achieve a target weight. After maturation, the pharmaceutical composition in the form of a hydrogel patch was prepared by cutting (70 cm²/sheet).

TABLE 1

| Purpose of mixing | Ingredient | Example 1 (wt %) |
|---|---|---|
| Main | Arnica | 0.70 (7% as 1X) |
| ingredient | | |
| Gel-forming | Sodium polyacrylate | 4.80 |
| agent | Polyacrylic acid | 2.00 |
| Dispersant | Glycerin | 39.00 |
| Cross- | Aluminum glycinate | 0.11 |
| linking | | |
| agent | | |
| Dissolution | 1,2-hexanediol | 0.30 |
| aid | Polysorbate 80 | 0.10 |
| Viscosity | Sodium carboxymethyl | 2.30 |
| modifier | cellulose | |
| Organic acid | Tartaric acid | 0.30 |
| Chelating | Sodium edetate | 0.085 |
| agent | | |
| Colorant | Titanium oxide | 0.20 |
| Water | Purified water | 50.105 |

| | |
|---|---|
| Total | 100.00 |

### Test Example 1. Study of Anti-Inflammatory Therapeutic Effects

A test for verifying the inflammation inhibitory therapeutic effect and the inflammatory disease marker inhibitory effect of Arnicare Gel® (Boiron Inc., France) and patches was performed using a carrageenan-induced inflammation mouse animal model. Arnicare Gel® is a commercially available product and serves as the control group in the present test.

### Experimental Method

1. Induction of edema using 1% carrageenan: 5 μL of 1% carrageenan in saline solution is injected into the right paw of the mouse, and 50 μL of saline is injected into the left paw of the mouse.
2. Drug application: According to the experimental flow chart below, Arnicare Gel® (Boiron Inc., France) and hydrogel patches are applied two to three times daily. For the drug application, the drug is administered daily from 9 a.m. for two days, and the gel is applied or the patch is attached immediately after the drug administration to improve the stability of the application. This is repeated, and the drug is additionally applied at 1 p.m. (6 p.m. for the gel) (two to three times daily). The patch is replaced at 9 a.m.
3. Termination of the experiment: On the 3rd day, the size of the edema is measured and the photographs of the edema are taken at hours 1, 2, and 3 after the administration of carrageenan and the final gel application or patch attachment. The shape is observed over time, and the blood is collected using the intraorbital vein blood collection method when changes are recognized, and the mouse is euthanized by cervical dislocation. The paw where mouse edema is induced and the contralateral paw for comparison are set as the observation targets. The skin on both paws is collected with a razor blade. mRNA sampling is performed therefrom. Reverse transcription polymerase chain reaction (RT-PCR) analysis for major inflammatory indicators is performed using mRNA.

### Experimental Results

In a mouse model in which edema was induced, a decrease in edema was confirmed in terms of the changes in the thickness in the two formulations, Arnicare Gel® (Boiron Inc., France) and the patch formulation. In addition, the results of the analysis of the tissue photographs collected after the experiment also confirmed the efficacy that the inflammatory cell infiltration was decreased more in the mouse tissues of the group treated with the patch formulation compared to the other group.

### Test Example 2. Study of Analgesic Therapeutic Effects

A hot plate pain alleviation validation test was conducted to analyze the analgesic effects of the gel formulation and hydrogel patch formulation containing the Arnica montana extract.

Experimental method
1. Selection of administration dose of gel and patch formulations (The selection was performed by converting the content of the commercially available preparations based on the human and mouse body areas.) (0.5 g of the gel, corresponding to 35 mg of the Arnica extract, was used, and 3.46 cm² of patch, containing 3.46 mg, was selected.)
2. The experiment was designed to include five groups (control group, 1-hour and 2-hour patch application groups, and 1-hour and 2-hour gel application groups), and the drug was applied to the left leg of the mice of all groups. The experiment was repeated eight times per group, and the information used for the result values was the average value for n=6 mice excluding the lowest and highest values.
3. During the hot plate experiment after drug application, the hot plate temperature was set to 55° C.
4. An acrylic cylinder was installed on the hot plate to restrict the movement of the mice.
5. The abnormal actions of the mice exposed to the hot plate for one minute was observed.
a. Flinching, licking, and jumping were defined as valid abnormal actions.
b. The number of times of each action was recorded.
c. The pain score is calculated by giving one point each for flinching and licking and two points for jumping.
6. The pain score (%) is calculated by comparing the treated and untreated legs in one mouse.

### Experimental Results

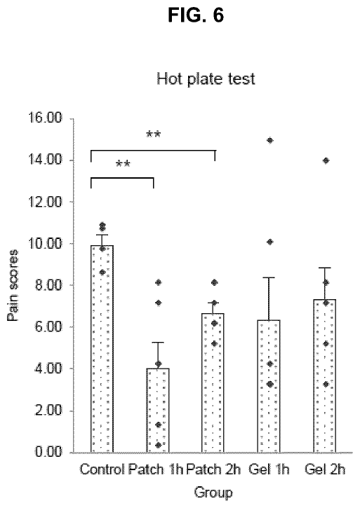

FIG. 6 shows the pain alleviating effect of the Arnica patch according to the pain score. When the pain score ratio of the treatment group to the control group is compared, it can be confirmed that there was a significant pain alleviating effect in the 1-hour and 2-hour patch formulation application groups.

TABLE 2

| | | | Patch | | |
|---|---|---|---|---|---|
| | Control | Patch 1 h | 2 h | Gel 1 h | Gel 2 h |
| Pain score | 9.667 | 1.000 | 7.000 | 3.000 | 7.000 |
| (n = 6) | 10.667 | 4.000 | 8.000 | 3.000 | 3.000 |
| | 8.500 | 7.000 | 8.000 | 3.000 | 5.000 |
| | 10.833 | 4.000 | 5.000 | 4.000 | 8.000 |
| | — | 8.000 | 6.000 | 10.000 | 14.000 |
| | — | 0.000 | 6.000 | 15.000 | 7.000 |
| Average pain | 9.917 | 4.000 | 6.667 | 6.333 | 7.333 |
| score | | | | | |
| Pain score | 1.000 | 0.403 | 0.672 | 0.639 | 0.739 |
| ratio | | | | | |
| Standard error | 0.538 | 1.291 | 0.494 | 2.060 | 1.520 |
| of mean (SEM) | | | | | |
| Standard | 1.076 | 3.162 | 1.211 | 5.046 | 3.724 |
| deviation (SD) | | | | | |

### Test Example 3. Formulation Stability

Other compositions were the same as Example 1, and only the contents of purified water and glycerin were adjusted to test the stability of the formulation.

Example 2: 30% by weight of water, 60% by weight of glycerin

Example 3: 40% by weight of water, 50% by weight of glycerin

Example 4: 60% by weight of water, 30% by weight of glycerin

Example 5: 70% by weight of water, 20% by weight of glycerin

As a result of the experiment, no patch was formed in Example 2, and the formulation stability failed to meet the standard. Examples 1, 3, and 4 exhibited excellent patch formation and adhesiveness, and were evaluated as Excellent in terms of skin irritation according to the Dermatest results. Example 5 formed a gel but had no adhesiveness, so it was not suitable as a patch.

The composition of the hydrogel or cataplasma patch formulation of the present invention can enable to fundamentally avoid the disadvantages of conventional cataplasmas and plasters, such as the problem of skin irritation, the toxicity problem due to residual solvents and/or unreacted monomers, and the problem of long-term crosslinking.

In addition, the composition of the hydrogel or cataplasma patch formulation of the present invention can evenly distribute a high concentration of drug while exhibiting excellent transdermal permeability without problems such as crystal precipitation during manufacturing and storage.

In addition, the present invention can provide an anti-inflammatory and analgesic composition containing a powder or extract of a fermented extract of Arnica as an active ingredient, which can alleviate pain through anti-inflammatory and analgesic properties in areas of sprains, bruises, muscle pain, joint pain, fracture pain, back pain, stiff shoulders, neuralgia, rheumatic pain, skin itchiness, insect bites, or chilblains.

1. A composition for a hydrogel or cataplasma patch containing an Arnica extract, comprising 30% to 70% by weight of water as a solvent based on the total composition, 20% to 60% by weight of a polyhydric alcohol based on the total composition, and a partially neutralized polyacrylic acid product as a polymer for forming a polyhydric alcohol gel, the composition being effective for suppressing inflammation, alleviating pain or alleviating hot plate pain.

2. The composition according to 1, wherein the Arnica extract is included in an amount of 0.7% to 1.7%.

3. The composition according to 1, wherein the partially neutralized polyacrylic acid product has a neutralization degree of 25% to 70% by mole.

4. The composition according to 1, wherein the polyhydric alcohol is at least one selected from the group consisting of glycerin, ethylene glycol, propylene glycol, dipropylene glycol, hexylene glycol, 1,2-hexanediol, and butylene glycol.

5. The composition according to 1, further comprising a cross-linking agent, wherein the crosslinking agent is at least one selected from the group consisting of a dried aluminum hydroxide gel, dihydroxyaluminum aminoacetate, aluminum hydroxide, aluminum sulfate, aluminum acetate, magnesium hydroxide, magnesium sulfate, magnesium acetate, calcium hydroxide, calcium carbonate, calcium chloride, calcium gluconate, calcium lactate, and calcium pantothenate.

6. The composition according to 1, further comprising an acid, wherein the acid is at least one selected from the group consisting of tartaric acid, acetic acid, propionic acid, isobutyric acid, caproic acid, caprylic acid, lactic acid, maleic acid, pyruvic acid, oxalic acid, succinic acid, tartaric acid, phthalic acid, salicylic acid, benzoic acid, acetylsalicylic acid, methanesulfonic acid, ethanesulfonic acid, propylsulfonic acid, and butanesulfonic acid.

7. The composition according to 1, further comprising a penetration enhancer, wherein the penetration enhancer is at least one selected from the group consisting of isopropyl myristate, lauryl alcohol, propylene glycol monolaurate, oleic acid, polyoxyethylene lauryl ether, diethylene glycol monoethyl ether, and isostearyl glyceryl ether, and is present in an amount of 0.5% to 10% by weight based on the total weight of the composition.

8. The composition according to 1, further comprising a chelating agent, wherein the chelating agent is edetic acid or a salt thereof, and is present in an amount of 0.2% to 3% by weight based on the total weight of the composition.
